(19) United States
(12) Patent Application Publication
(10) Pub. No.: US 2023/0043519 A1
(43) Pub. Date: Feb. 9, 2023

(54) METHOD AND SYSTEM FOR SUPPLEMENTING SCAN DATA BY USING LIBRARY DATA

(71) Applicant: MEDIT CORP., Seoul (KR)

(72) Inventor: Won Hoon CHOI, Seoul (KR)

(73) Assignee: MEDIT CORP., Seoul (KR)

(21) Appl. No.: 17/970,689

(22) Filed: Oct. 21, 2022

Related U.S. Application Data
(63) Continuation of application No. PCT/KR2021/005082, filed on Apr. 22, 2021.

(30) Foreign Application Priority Data
Apr. 24, 2020 (KR) 10-2020-0050161
Apr. 22, 2021 (KR) 10-2021-0052195

(51) Int. Cl. A61C 13/00; A61C 9/00

(52) U.S. Cl. CPC A61C 13/0004; A61C 9/0046

(57) ABSTRACT

A method for supplementing scan data by using library data according to the present invention comprises: a scan step of acquiring scan data of a subject including a structure; a step of selecting library model data corresponding to the structure; and a step of post-processing the scan data, wherein the library model data is added at the step of post-processing of the scan data and is post-processed together with the scan data. Accordingly, the scan data in which distortion or a data gap has occurred is supplemented by the library model data, and thus a user acquires a highly reliable three-dimensional model.

### TECHNICAL FIELD

The present disclosure relates to a method and system for supplementing scan data using library data. More particularly, the present disclosure relates to a method and system for supplementing scan data using library data, wherein library data is added in a post-processing process when scan data is post-processed.

### BACKGROUND ART

A three-dimensional (3-D) scanning technology is currently used in various fields, and has been in the spotlight that the 3-D scanning technology will also be consistently used in the industry fields in the future because the accuracy and rapidity of the scanning technology is continuously improved.

In performing 3-D scanning, if a person (user) who performs a scan does not scan a subject with a sufficient time or amount, scan data relating to a part corresponding to a part of the subject is not generated and remains in a blank state. Particularly, it may be difficult to obtain, through a scan according to light radiation, data of a part that is made of a metal material having severe light reflection. It is important for the user to closely scan the subject so that insufficient data is minimized after performing 3-D scanning. However, there may be a case in which scan data is insufficiently obtained due to scan limits, a degree of fatigue of the user may rise, and user convenience may be caused.

In order to supplement such incompleteness in obtaining data, there is a need for a method capable of filling an imperfect part of data that is obtained by a scan or correcting an error part of the data by using 3-D-designed data (e.g., 3-D drawing data).

### DISCLOSURE

### Technical Problem

The present disclosure provides a method of supplementing scan data using library data, which can supplement scan data that is obtained in a scan step with previously mounted library model data.

Furthermore, the present disclosure provides a system for supplementing scan data using library data, which can supplement scan data with library model data by performing a method of supplementing scan data using library data.

Objects of the present disclosure are not limited to the aforementioned object, and the other objects not described above may be evidently understood from the following description by those skilled in the art.

### Technical Solution

A method of supplementing scan data using library data according to the present disclosure includes a scan step of obtaining scan data of a subject including a structure, a step of selecting library model data corresponding to the structure, and a step of post-processing the scan data, wherein the library model data is added in the step of post-processing and is post-processed along with the scan data.

Furthermore, the library model data added in the step of post-processing may be aligned with at least some of the scan data.

Furthermore, the at least some of the scan data may be aligned on the basis of the library model data.

Furthermore, in a three-dimensional (3-D) model of the structure, a data blank of the structure that has not been scanned in the scan step may be supplemented with the library model data.

Meanwhile, a method of supplementing scan data using library data according to another embodiment of the present disclosure includes a scan step of obtaining scan data of a subject including a structure, and an alignment step of generating a three-dimensional (3-D) model of a subject by aligning the scan data with library model data corresponding to the structure.

Furthermore, the subject may be at least one selected from a group including a mouth of a patient, a negative model of the mouth, and a positive model of the mouth.

Furthermore, the structure may be at least any one selected among prosthetic appliances including a scanbody or an abutment inserted into the subject.

Furthermore, the alignment step may include a local alignment step of sequentially aligning the scan data input in the scan step, and a global alignment step of generally aligning at least some of the scan data which is input with the library model data after the local alignment step is terminated.

Furthermore, a real-time 3-D model of the subject may be generated in the local alignment step, and the real-time 3-D model may be reconstructed in the global alignment step.

Furthermore, the library model data may be selected before the alignment step.

Furthermore, the library model data may be selected before or after the scan step.

Furthermore, the library model data may be selected before the global alignment step.

Furthermore, the library model data may be selected before the scan step or after the local alignment step.

Furthermore, the library model data may be automatically or manually selected.

Furthermore, when the library model data is manually selected, the library model data may be selected in a library interface in response to an input from a user.

Meanwhile, a system for supplementing scan data using library data according to the present disclosure may include a scan unit configured to obtain scan data of a subject including a structure, and a controller configured to generate a three-dimensional (3-D) model of the subject by aligning the obtained scan data with library model data corresponding to the structure.

Furthermore, the controller may be embedded and formed in the scan unit or may be formed to be spaced apart from the scan unit.

Furthermore, the scan unit may be a handheld scanner or a table scanner.

Furthermore, the scan unit may include at least one camera configured to accommodate light reflected by the subject, and an imaging sensor telecommunicationally connected to the camera and configured to obtain a two-dimensional (2-D) image of the subject.

Furthermore, the controller may include a three-dimensional (3-D) conversion unit configured to convert the 2-D image of the subject into 3-D data, and an alignment unit configured to align the 3-D data.

Furthermore, the alignment unit may include a local alignment unit configured to perform sequential alignment on the 3-D data that are consecutive to each other, and a global alignment unit configured to generally align the 3-D data with the library model data after the sequential alignment performed by the local alignment unit.

Furthermore, the controller may further include a library selection unit configured to select the library model data, and the library model data selected by the library selection unit may supplement the scan data obtained by the scan unit.

### Advantageous Effects

There is an advantage in that by using the method and system for supplementing scan data using library data according to the present disclosure, data having high reliability can be provided upon fabrication of a prosthetic cure by supplementing insufficient scan data with previously mounted library model data although the insufficient scan data is obtained.

### DESCRIPTION OF DRAWINGS

FIG. 1 is a schematic flowchart of a method of supplementing scan data using library data according to the present disclosure.

FIG. 2 is a diagram in which scan data is displayed in a scan interface before scan data is obtained and supplemented.

FIG. 3 is a diagram in which the results of measurement of the size of a prosthetic cure obtained based on scan data are displayed in the scan interface.

FIG. 4 is a diagram in which scan data is displayed in the scan interface before the scan data is obtained and supplemented.

FIG. 5 is a diagram in which a process of supplementing a data blank or a part having low data density by disposing library model data on scan data in a method of supplementing scan data using library data according to the present disclosure is displayed.

FIG. 6 is a diagram illustrating a deviation between scan data before library data is used and supplementation data supplemented from the scan data by using library model data.

FIG. 7 is a schematic diagram of a construction of a system for supplementing scan data using library data according to the present disclosure.

### DESCRIPTION REFERENCE NUMERALS

S10: scan step S11: image acquisition step

S12: 3-D conversion step S20: alignment step

S21: local alignment step S22: global alignment step

S30: library data selection step

1: scan data

10: gum

20: tooth

21, 21a, 21b, 21c, 21d: structure (or library model)

D: internal hole D′: inaccurately scanned internal hole

B: data blank LD: library model data

100: system for supplementing scan data

110: scan unit 120: controller

121: database unit 122: 3-D conversion unit

123: alignment unit 123a: local alignment unit

123b: global alignment unit 124: library selection unit

130: output unit 140: communication unit

### Best Mode

Hereinafter, some embodiments of the present disclosure will be described in detail with reference to exemplary drawings. In adding reference numerals to the elements of each drawing, it should be noted that the same elements have the same reference numerals as much as possible even if they are displayed in different drawings. Furthermore, in describing embodiments of the present disclosure, when it is determined that a detailed description of the related well-known configuration or function hinders understanding of an embodiment of the present disclosure, the detailed description thereof will be omitted.

Furthermore, in describing elements of an embodiment of the present disclosure, terms, such as a first, a second, A, B, (a), and (b), may be used. Such terms are used only to distinguish one component from another component, and the essence, order, or sequence of a corresponding component is not limited by the terms. All terms used herein, including technical or scientific terms, have the same meanings as those commonly understood by a person having ordinary knowledge in the art to which the present disclosure pertains, unless defined otherwise in the specification. Terms, such as those commonly used and defined in dictionaries, should be construed as having the same meanings as those in the context of a related technology, and are not construed as being ideal or excessively formal unless explicitly defined otherwise in the specification.

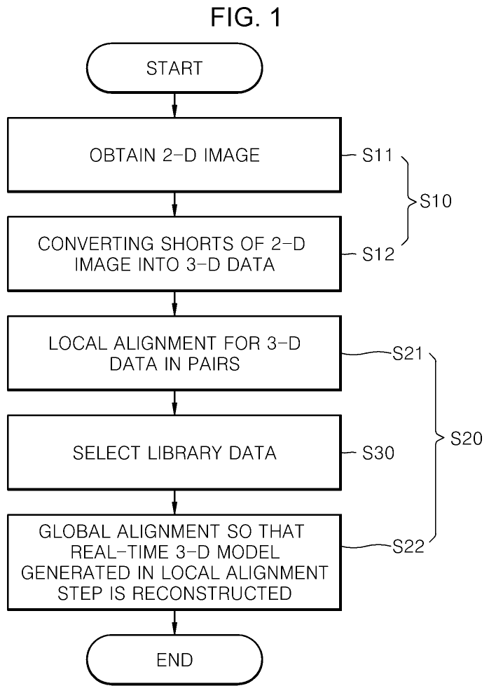

FIG. 1 is a schematic flowchart of a method of supplementing scan data using library data according to the present disclosure.

Referring to FIG. 1, the method of supplementing scan data using library data according to the present disclosure may include a scan step S10 of obtaining, by a scanner, scan data 1 by scanning a subject including a structure. In this case, the subject may be the inside of the actual mouth of a patient, that is, the object of treatment, for the purpose of the present disclosure that requires scan data, such as a tooth 20, a gum 10, and a jawbone. However, the subject is not essentially limited to the inside of the actual mouth of a patient, and the subject may be an oral model (plaster cast) for testing the insertion depth and insertion angle of a structure before the structure is inserted into the mouth of a patient. The oral model may be a negative model, that is, an impression obtained by performing impression taking by using alginate, etc. or may be a positive model obtained by filling a negative model with a material such as plaster. Accordingly, the scan data 1 may be digital data of the negative or positive model that is imitated from a real thing within the mouth of a patient or the inside of the mouth. The structure may be at least one of an abutment that is inserted into a subject for an implant or crown treatment or a scanbody by which the insertion depth and direction of a fixture inserted into the gum 10 can be checked.

The scan step S10 is performed through the scanner which is driven by a user. In this case, a different type of scanner may be used depending on the type of subject. For example, if the subject is an oral model subjected to impression taking, the scanner that is driven by a user may be a table scanner which includes an internal space therein on which a subject can be held and which obtains scan data by photographing the subject through a camera disposed within the scanner while tilting or rotating the subject. If the subject is the inside of an actual mouth of a patient, the scanner that is driven by a user may be a handheld type intraoral scanner capable of actively adjusting a scan distance and scan angle thereof with respect to a subject depending on user needs in a way that the scanner is directly inserted into or drawn out from the mouth of the patient by the grip of a hand of the user. In this case, if the scanner that is driven by a user is the handheld type intraoral scanner, the subject may be at least one of an actual mouth of a patient, a negative model, and a positive model. That is, if the subject is any one of the positive model corresponding to an oral model and the negative model corresponding to an impression, the scanner that performs the scan step S10 may be the table scanner or the handheld type intraoral scanner.

The scan step S10 may include an image acquisition step S11 of obtaining a two-dimensional (2-D) image through at least one camera that is disposed within the scanner and an imaging sensor that is telecommunicationally connected to the camera. The scanner may include a case in which a tip is formed at one end thereof so that the scanner may be inserted into and drawn out from the mouth. The at least one camera may be disposed within the case so that light that enters the case may be accommodated therein. In this case, the camera may be a single camera or two or more cameras. When light is accommodated within the camera, the light may be formed in the form of electronic image information (data) by the imaging sensor that is telecommunicationally connected to the camera. The imaging sensor replaces a conventional film, and may be a CCD sensor or a CMOS sensor, for example. However, the imaging sensor is not essentially limited to the CCD sensor or the CMOS sensor, and the imaging sensor may be a color imaging sensor if necessary. The image data obtained in the image acquisition step S11 may be 2-D image data on a plane. The camera and the imaging sensor embedded in the scanner obtain the image data by continuously photographing the subject in a process of the scanner performing scans.

Furthermore, the scan step S10 may further include a three-dimensional (3-D) conversion step S12 of converting the 2-D image data, obtained in the image acquisition step S11, into 3-D data having a volume. In order to convert the 2-D image data into the 3-D data, a plurality of shots for the 2-D image data may be used. In order to obtain depth information, etc. that are necessary to convert the 2-D image data into the 3-D data, a light radiation device such as a light projector disposed within the scanner may radiate, to the subject, structure light having a specific pattern. In this case, the light radiated by the light radiation device may have various wavelength regions. Light having a wavelength, which may sharply obtain an image of the subject while not damaging the inside of the mouth of the patient, may be used as the light radiated by the light radiation device. For example, the light radiated by the light radiation device may be light having a visible ray region. The 3-D data that has been obtained as described above may be subsequently used to finally generate a 3-D model that expresses the subject.

The 3-D conversion step S12 of converting the 2-D image data into the 3-D data may be performed by a 3-D conversion unit of a controller that is electrically connected to the scanner. However, the 3-D conversion step S12 is not limited to being essentially performed by the controller, and the 3-D conversion step S12 may be performed by a processor that has an arithmetic capacity capable of performing a 3-D conversion process and that is embedded in the scanner. Alternatively, in a system for supplementing scan data using library data, which is described later, the entire controller may be constructed to be embedded in the scanner (scan unit), and the controller may perform all types of operation processing within the scan unit.

Meanwhile, an alignment step S20 of generating a 3-D model of the subject by aligning overlap parts of a plurality of 3-D data that expresses partial shape information of the entire subject as described above may be performed. In this case, the alignment step S20 may be performed as one or more steps. For example, the alignment step S20 may include a local alignment step S21 of forming a real-time 3-D model by sequentially aligning the scan data (the 2-D image data or the 3-D data), input and obtained in the scan step S10, in pairs. In this case, a method of performing the local alignment may be performed by using an iterative closest point (ICP) method, that is, one of point cloud alignments. The real-time 3-D model that expresses the subject may be generated through the local alignment step S21.

The local alignment step S21 may be performed through an alignment unit (more particularly, a local alignment unit), that is, one component of the controller. However, if the 3-D conversion step S12 is performed by the processor embedded in the scanner, the local alignment step S21 may be performed by at least one of the processor embedded in the scanner and the alignment unit of the controller.

When the real-time 3-D model that expresses the subject is generated through the local alignment step S21, library model data corresponding to the structure that has been inserted into the subject may be selected (a library selection step S30). That is, in the library selection step S30, the library model data may be provided to the user in a way that a library selection unit, that is, one component of the controller, controls an output unit. For example, the controller may output a library interface through the output unit. The user may select at least one of the library model data that is displayed in the library interface through an input unit (not illustrated).

The “library” may be data that is embedded in a program or application in which the method of supplementing scan data according to the present disclosure is performed. Library model data LD means the existing embedded model data. In this case, the library model data LD may include various objects. For example, the library model data LD may be scanbody model data which may be inserted into the inside of an actual mouth of a patient or a part (more particularly, a gum) of an oral model. For example, if a structure that is inserted into the tooth 20 is a scanbody, the library model data LD may be implemented in the form of a model including 3-D shape information, such as flexure information of a surface of the scanbody, color information of the surface, and axis information. Such library model data LD may be formed by using a computer aided design (CAD) tool, but the present disclosure is not limited thereto.

The library model data LD may be manually selected in response to an input from a user in a library interface that is disposed to be isolated from a scan interface corresponding to a work space of a program as described above. However, the library model data LD may not be always manually selected in response to an input from a user. The library model data LD may be automatically selected based on the scan data 1 that is obtained by scanning the subject including the structure in the scan step S10. When a scan is performed in the scan step S10, the controller may recognize the structure having a shape or material different from that of the gum or the tooth by the scan step S10 performed by the scanner. In the library selection step S30, the library model data LD having corresponding information may be automatically selected by the controller (e.g., the library selection unit) based on the material, the shape, etc. of the structure recognized in the scan step S10.

As described above, the library selection step S30 has been described as being performed after the local alignment step S21, but the present disclosure is not essentially limited thereto. For example, a process of selecting the library model data LD in the library selection step S30 may be performed before the scan step S10 or may be performed between the scan step S10 and the alignment step S20 (i.e., before the alignment step S20 after the scan step S10). In a different example, the library selection step S30 may be performed after the aforementioned local alignment step S21 and before a post-processing step (e.g., the global alignment step S22) to be described later, that is, between the local alignment step S21 and the post-processing step. However, due to the nature of the present disclosure, the library selection step S30 may be performed before the post-processing step is performed, and the library model data LD may be selected so that the library model data LD is aligned with the scan data 1 when the post-processing step is performed.

Meanwhile, the library model data LD selected in the library data selection step S30 may have shape information corresponding to a location of a tooth into which each library model is inserted. In this case, the shape information may include flexure information of a surface, color information of the surface, axis information, etc. as described above. Particularly, if the structure is a scanbody, information, such as a center axis, height, or direction of the library model data, may be included in the shape information. In this case, the library model data LD includes size and direction information, etc. of the existing structure, and may be more close to a real size than the size of the scan data 1 that is obtained due to an insufficient scan in the scan step S10. Particularly, the size of the existing structure may act as a reference for the size, a shape, etc. of a new structure because the structure is rarely worn away when the structure is inserted into the subject.

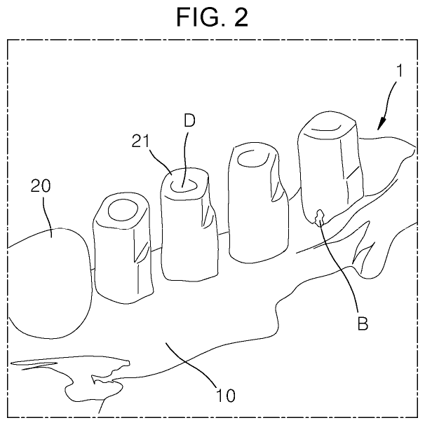

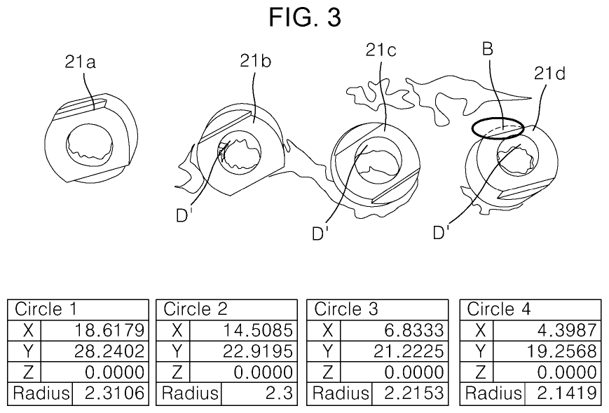

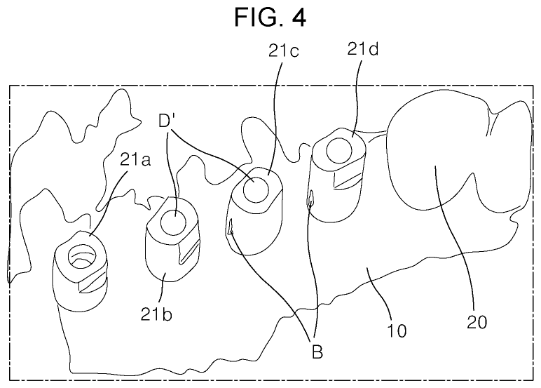

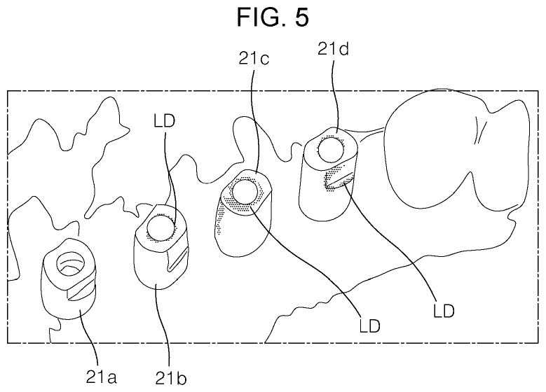

FIG. 2 is a diagram in which scan data is displayed in a scan interface before the scan data 1 is obtained and supplemented. FIG. 3 is a diagram in which the results of measurement of the size of the scanbody obtained based on the scan data 1 are displayed in the scan interface. Furthermore, FIG. 4 is a diagram in which the scan data 1 is displayed in the scan interface before the scan data 1 is obtained and supplemented. FIG. 5 is a diagram in which a process of supplementing a data blank B or a part having low data density by disposing the library model data LD on the scan data 1 in a method of supplementing scan data using library data according to the present disclosure is displayed.

Referring to FIGS. 2 to 5, in the local alignment step S21, the distortion of the scan data 1 may occur because parts overlapped between the scan data 1 are not sufficiently aligned, with respect to a part having low data density because obtained scan data is not sufficient due to the reflection of light of a scanbody made of a metal material. Such distortion of the scan data 1 causes an axial direction error in addition to an insufficient shape expression (a data blank). In addition, if matching with the library model data LD is attempted, a margin error with an abutment, a surrounding tooth, etc. inevitably occurs when an prosthetic appliance, such as a crown, is fabricated based on erroneous matching results, such as that the matching is not smoothly performed or is performed in an inclined axial direction due to the insufficient shape expression.

In order to solve the aforementioned errors, in the present disclosure, at least some of the scan data 1 that has been distorted as described above may be supplemented based on shape information of the library model data LD. In this case, to supplement the scan data 1 with the library model data LD may mean that real-time 3-D model data of the scan data 1 and the library model data LD, which are obtained in the 3-D conversion step S12 and the local alignment step S21, are aligned with each other. This is for further improving the reliability of data of a part into which a prosthetic cure, such as a scanbody, has been inserted. If necessary, the scan data 1 having low data density may be filled with the library model data LD with respect to a structure, such as a scanbody, because the library model data LD has high accuracy in terms of the size thereof.

Referring to FIGS. 2 and 3, the state in which the scan data 1 is not closely expressed and the data blank B also occurs because the scan data 1 for at least a part of an outer circumference surface of a structure 21 that is inserted into the gum 10 is not completely obtained may be seen. Furthermore, it may be seen that when the radius (the diameter may be measured according to circumstances) of an internal hole D of the structure 21 is measured, the radius of each structure 21 is different due to an inaccurately scanned internal hole D′. For example, the inaccurately scanned internal hole D′ may appear in at least one of a first structure 21a, a second structure 21b, a third structure 21c, and a fourth structure 21d in FIG. 3. In such a case, a correction, deletion and/or supplementation may be performed on a part that has been erroneously scanned or a part that has not been scanned due to carelessness, etc. of a user.

Referring to FIG. 4, if only the scan data 1 is displayed in the scan interface, the inaccurately scanned the internal hole D′ and the data blank B are found. Referring to FIG. 5, the library model data LD may be aligned with the scan data 1. The library model data LD may be displayed in a color different from that of the scan data 1, in a part that belongs to the scan data 1 and that has low data density.

After the real-time 3-D model is formed by sequentially aligning consecutive 3-D data in pairs in the aforementioned local alignment step S21, in order to supplement inaccurate data of the real-time 3-D model, the controller (e.g., the global alignment unit of the alignment unit) may finally perform a post-processing step so that the scan data 1 is generally aligned and reconstructed in the form of the final 3-D model. For example, the post-processing step may be a global alignment step S22 of reconstructing the real-time 3-D model that has been locally aligned as a 3-D model by generally aligning the real-time 3-D model. In order to finally generate the 3-D model, the controller may perform the global alignment step S22 as the last step so that the real-time 3-D model is reconstructed as the 3-D model. For example, the global alignment step S22 may be performed by selecting a complete button formed in a user interface after the scanning of the subject is completed. Accordingly, as the scan data 1 is supplemented with the library model data LD and the final 3-D model of the subject is generated, there are advantages in that the reliability of the 3-D model that represents the subject can be improved, a precise prosthetic cure can be fabricated and provided to a patient, and resultantly optimum treatment can be provided to the patient.

When the global alignment step S22 is performed, the library model data LD selected in the library selection step S30 may be used along with at least some of the scan data (in this case, the scan data 1 may mean 3-D data). That is, in the global alignment step S22, when the 3-D model is generally reconstructed, the library model data LD may supplement the data blank B or a part in which noise occurs. For example, in the aforementioned local alignment step S21, if a reference point overlapped between 3-D data is not sufficient, the distortion of scan data or the data blank B occurs. In this case, if the global alignment step S22 is performed by adding the library model data LD selected in the library selection step S30 between the 3-D data, the reference point at which the 3-D data and the library model data LD may be overlapped, respectively, is increased. Accordingly, although a real-time 3-D model has been distorted and formed differently from the subject in the local alignment step S21, scan data that has been distorted (contracted) or a data blank when a global alignment process, that is, a post-processing process, is performed can be aligned and/or supplemented again based on the library model data LD. As a result, a 3-D model having high reliability can be obtained.

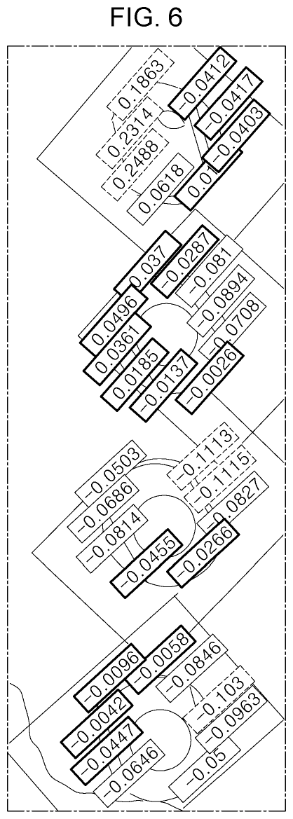

FIG. 6 is a diagram illustrating a deviation between scan data before library data is used and supplementation data supplemented from the scan data by using library model data.

FIG. 6 illustrates a deviation between information of only the scan data in FIG. 4 and information of supplementation data generated from the library model data in FIG. 5 by supplementing the scan data. Accordingly, deviations from 6 micrometers to 200 micrometers appear. As described above, a part having a large deviation value is a part in which scan data is insufficient due to an inaccurate scan or has been erroneously obtained. It can be easily checked through FIG. 6 that in order to obtain a more precise 3-D model, it is profitable to supplement scan data by using library model data.

Hereinafter, a system for supplementing scan data using library data according to the present disclosure is described.

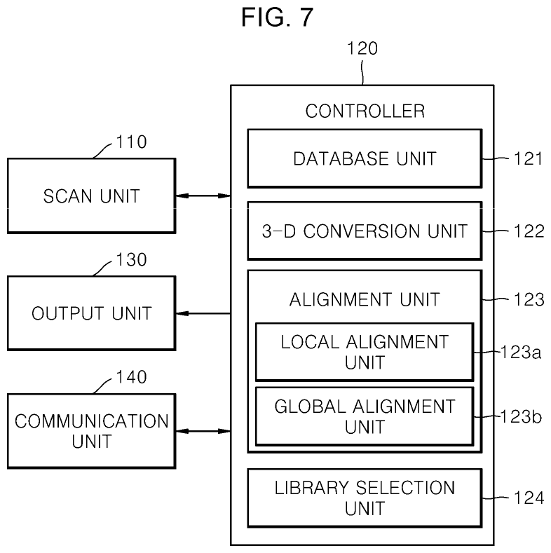

FIG. 7 is a schematic diagram of a construction of the system for supplementing scan data using library data according to the present disclosure.

Referring to FIG. 7, the system 100 for supplementing scan data using library data according to the present disclosure may include a scan unit 110 obtaining scan data of a subject including a structure, a controller 120 generating a 3-D model of the subject by aligning the obtained scan data and library model data corresponding to the structure with each other, and an output unit 130 outputting scan data by receiving a control signal from the controller 120.

The scan unit 110 may obtain scan data by scanning a subject. In this case, the subject may include a structure, such as an abutment or a scanbody. Furthermore, the subject may be the inside of an actual mouth of a patient into which a structure has been inserted or may be plaster cast or an impression that is obtained by performing impression taking on the mouth of a patient. The scan unit 110 may be a 3-D scanner for scanning such a subject. The 3-D scanner may be a table scanner or a handheld type intraoral scanner for finally forming a 3-D model based on an obtained image. The “scanner” mentioned when the aforementioned method of supplementing scan data using library data is described may be interpreted as corresponding to the scan unit 110 of the system for supplementing scan data using library data according to an embodiment of the present disclosure.

Furthermore, the scan unit 110 may include at least one camera for receiving light that is reflected by a subject and an imaging sensor telecommunicationally connected to the camera. The camera may receive, from a camera lens, light reflected by a subject and focuses the light on the imaging sensor that plays a role as a retina or a film. The imaging sensor may generate 2-D image data by analyzing the light. The generated 2-D image data may be stored in a database unit 121. Alternatively, the generated 2-D data may be stored in a separate storage unit (not illustrated) embedded in the scan unit 110.

In order for the 2-D image data that is obtained by a scan process of the scan unit 110 to be formed in the form of a 3-D model, the 2-D image data may be converted into 3-D data by a 3-D conversion unit 122 within the controller 120. In order for the 2-D image data to be converted into the 3-D data, the scan unit 110 may radiate, to the subject, structure light having a specific pattern. The 3-D conversion unit 122 may convert, into the 3-D data having a volume, the 2-D image data obtained by analyzing 2-D image data having a pattern. However, if a processor capable of a 3-D operation is embedded in the scan unit 110, the processor embedded in the scan unit 110 may convert the 2-D image data into the 3-D data.

The aforementioned scan unit 110 may be a handheld type scanner for obtaining data by partially consecutively scanning a subject. Particularly, the handheld type scanner may be an intraoral scanner that is used for dental treatment purposes. However, a construction of the scan unit 110 is not limited to the intraoral scanner, and may be a table scanner for holding, in a tray formed within the scan unit 110, an oral model that is obtained by performing impression taking on the mouth of a patient and then obtaining data by generally scanning the oral model according to circumstances.

The controller 120 may further include an alignment unit 123 for aligning the 3-D data converted by the 3-D conversion unit 122. The alignment unit 123 may list and merge the 3-D data through alignment between data that are partially or generally overlapped. Preferably, at least two alignments are performed by the alignment unit 123. For example, the alignment unit 123 may include a local alignment unit 123a for sequentially performing local alignments on consecutive 3-D data obtained by the 3-D conversion unit 122 and a global alignment unit 123b for performing general alignment on all 3-D data on which local alignment has been performed. As the global alignment unit 123b performs global alignment, the 3-D data may be formed (reconstructed) in the form of the final 3-D model, and the reliability of data can be improved. The local alignment process of the local alignment unit 123a and the global alignment process of the global alignment unit 123b have been described in related steps of the method of supplementing scan data using library data according to an embodiment of the present disclosure, and a detailed description thereof is omitted.

In order to form the final 3-D model by the global alignment unit 123b, a library selection unit 124 enables the library model data to be used in a process of supplementing the 3-D model, so that the finer final 3-D model can be obtained. The library selection unit 124 may select the library model data of the structure that is embedded in the library of the database unit 121. In this case, the library model data may mean 3-D design drawing data of the structure (means a prosthetic cure such as an abutment or a scanbody) as described above. The library model data may be selected by the manual selection of the user or by automatic selection based on information of the structure that is obtained by the scanning of the subject including the structure in the scan process of the scan unit 110. The selected library model data may be preferably subjected to the global alignment process along with the 3-D data on which the local alignment process has been completed by the local alignment unit 123a. Accordingly, insufficient scanning of the scan data (data obtained by the scanning) can be supplemented with the library model data.

The controller 120 may be formed in a form mounted on the scan unit 110, that is, so that the controller 120 is embedded in the case of the scan unit 110. Alternatively, the controller 120 is not formed within the scan unit 110, but may be formed to be spaced apart from the scan unit 110 and telecommunicationally connected to the scan unit 110. In this case, if the scan unit 110 and the controller 120 are formed to be spaced apart from each other, the scan data obtained from the scan unit 110 may be transmitted to the controller 120 connected to the scan unit 110 in wired and wireless manners, and may be used to generate and align the 3-D data, to select the library, to supplement data, etc.

Such a process of generating the 3-D data by the 3-D conversion unit 122, an alignment process by the alignment unit 123, etc. may be output to the output unit 130 in response to a control signal applied by the controller 120. The output unit 130 may include any means capable of outputting the 3-D conversion or alignment process to the user, but may be preferably a monitor device capable of displaying the aforementioned process. The user may check the scan data and the library model data through the output unit 130, and may perform an additional scan on a corresponding part if the additional scan is required or may select proper library model data that is necessary to supplement the scan data.

Furthermore, a communication unit 140 may receive or transmit, from or to another system, etc., information such as 3-D conversion, alignment, and library model data selection that are performed by the controller. A scan and supplemented data of the system for supplementing scan data according to the present disclosure or related information may be shared with another system through the communication unit 140. Required information may be shared with another system through the communication unit 140.

The above description is merely a description of the technical spirit of the present disclosure, and those skilled in the art may change and modify the present disclosure in various ways without departing from the essential characteristic of the present disclosure.

Accordingly, the embodiments described in the present disclosure should not be construed as limiting the technical spirit of the present disclosure, but should be construed as describing the technical spirit of the present disclosure. The technical spirit of the present disclosure is not restricted by the embodiments. The range of protection of the present disclosure should be construed based on the following claims, and all of technical spirits within an equivalent range of the present disclosure should be construed as being included in the scope of rights of the present disclosure.

### INDUSTRIAL APPLICABILITY

The present disclosure provides the method of supplementing scan data using library data, which supplements scan data that has been distorted in a post-processing process by using previously mounted library model data when forming a 3-D model by obtaining scan data of a subject.

1. A method of supplementing scan data using library data, the method comprising:
a scan step of obtaining scan data of a subject comprising a structure through a scanner;
a step of selecting, by a controller, library model data corresponding to the structure; and
a step of post-processing, by the controller, the scan data,
wherein the library model data is added in the step of post-processing and is post-processed along with the scan data.

2. The method of claim 1, wherein the library model data added in the step of post-processing is aligned with at least some of the scan data.

3. The method of claim 2, wherein the at least some of the scan data is aligned on the basis of the library model data.

4. The method of claim 1, wherein in a three-dimensional (3-D) model of the structure, a data blank of the structure that has not been scanned in the scan step is supplemented with the library model data.

5. A method of supplementing scan data using library data, the method comprising:
a scan step of obtaining scan data of a subject comprising a structure through a scanner; and
an alignment step of generating, by a controller, a three-dimensional (3-D) model of a subject by aligning the scan data with library model data corresponding to the structure.

6. The method of claim 5, wherein the subject is at least one selected from a group comprising a mouth of a patient, a negative model of the mouth, and a positive model of the mouth.

7. The method of claim 5, wherein the structure is at least any one selected among prosthetic appliances comprising a scanbody or an abutment inserted into the subject.

8. The method of claim 5, wherein the alignment step comprises:
a local alignment step of sequentially aligning the scan data input in the scan step; and
a global alignment step of generally aligning at least some of the scan data which is input with the library model data after the local alignment step is terminated.

9. The method of claim 8, wherein:
a real-time 3-D model of the subject is generated in the local alignment step, and
the real-time 3-D model is reconstructed in the global alignment step.

10. The method of claim 5, wherein the library model data is selected before the alignment step.

11. The method of claim 10, wherein the library model data is selected before or after the scan step.

12. The method of claim 8, wherein the library model data is selected before the global alignment step.

13. The method of claim 12, wherein the library model data is selected before the scan step or after the local alignment step.

14. The method of claim 5, wherein the library model data is automatically or manually selected.

15. The method of claim 14, wherein when the library model data is manually selected, the library model data is selected in a library interface in response to an input from a user.

16. A system for supplementing scan data using library data, the system comprising:
a scan unit configured to obtain scan data of a subject comprising a structure; and
a controller configured to generate a three-dimensional (3-D) model of the subject by aligning the obtained scan data with library model data corresponding to the structure.

17. The system of claim 16, wherein the scan unit comprises:
at least one camera configured to accommodate light reflected by the subject, and
an imaging sensor telecommunicationally connected to the camera and configured to obtain a two-dimensional (2-D) image of the subject.

18. The system of claim 17, wherein the controller comprises:
a three-dimensional (3-D) conversion unit configured to convert the 2-D image of the subject into 3-D data, and
an alignment unit configured to align the 3-D data.

19. The system of claim 18, wherein the alignment unit comprises:
a local alignment unit configured to perform sequential alignment on the 3-D data that are consecutive to each other, and
a global alignment unit configured to generally align the 3-D data with the library model data after the sequential alignment performed by the local alignment unit.

20. The system of claim 19, wherein:
the controller further comprises a library selection unit configured to select the library model data, and
the library model data selected by the library selection unit supplements the scan data obtained by the scan unit.
